(19) United States
(12) Patent Application Publication
(10) Pub. No.: US 2025/0127426 A1
(43) Pub. Date: Apr. 24, 2025

(54) MOTION STATE MONITORING SYSTEM, METHOD FOR CONTROLLING THE SAME, AND NON-TRANSITORY COMPUTER READABLE MEDIUM STORING CONTROL PROGRAM

(71) Applicant: Toyota Jidosha Kabushiki Kaisha, Toyota-shi (JP)

(72) Inventors: Makoto KOBAYASHI, Nisshin-shi (JP); Hiroto Ichikawa, Seto-shi (JP)

(73) Assignee: Toyota Jidosha Kabushiki Kaisha, Toyota-shi (JP)

(21) Appl. No.: 18/818,898

(22) Filed: Aug. 29, 2024

(30) Foreign Application Priority Data
Oct. 24, 2023 (JP) 2023-182859

(51) Int. Cl. A61B 5/11; A61B 5/05

(52) U.S. Cl. CPC A61B 5/1126; A61B 5/05; A61B 2560/0223; A61B 2562/0223

(57) ABSTRACT

A motion state monitoring system according to the present disclosure is a motion state monitoring system configured to monitor a specified motion to be monitored of a subject by two or more sensors selected from among a plurality of sensors made to correspond to a plurality of respective body parts of the subject, the motion state monitoring system including: an arrangement mechanism in which the two or more sensors are arranged in a known direction; a calibration execution unit configured to perform a calibration of the two or more sensors arranged in the known direction by the arrangement mechanism; and a monitoring result generation unit configured to generate a result of the monitoring of the motion to be monitored of the subject based on results of detection by the two or more sensors attached to the subject after the calibration is completed.

### CROSS REFERENCE TO RELATED APPLICATIONS

This application is based upon and claims the benefit of priority from Japanese patent application No. 2023-182859, filed on Oct. 24, 2023, the disclosure of which is incorporated herein in its entirety by reference.

### BACKGROUND

The present disclosure relates to a motion state monitoring system, a method for controlling the same, and a control program.

Patent Literature 1 discloses a motion state monitoring system that monitors a motion state of a subject based on a result of detection by a plurality of sensors attached to a plurality of respective body parts of a body of the subject. Patent Literature 1 also discloses a calibration of a plurality of sensors.

Patent Literature 1: Japanese Unexamined Patent Application Publication No. 2022-34448

### SUMMARY

The motion state monitoring system that monitors a motion state of a subject as disclosed in the related art is required to accurately perform a calibration of a plurality of sensors used to monitor a motion to be monitored of a subject in order to accurately monitor the motion to be monitored of the subject.

The present disclosure has been made in view of the aforementioned circumstances and an object thereof is to provide a motion state monitoring system, a method for controlling the same, and a control program that are capable of accurately monitoring a motion state of a subject.

A motion state monitoring system according to the present disclosure is a motion state monitoring system configured to monitor a specified motion to be monitored of a subject by two or more sensors selected from among a plurality of sensors made to correspond to a plurality of respective body parts of the subject, the motion state monitoring system including: an arrangement mechanism in which the two or more sensors are arranged in a known direction; a calibration execution unit configured to perform a calibration of the two or more sensors arranged in the known direction by the arrangement mechanism; and a monitoring result generation unit configured to generate a result of the monitoring of the motion to be monitored of the subject based on results of detection by the two or more sensors attached to the subject after the calibration is completed. The above-described motion state monitoring system performs a calibration of a plurality of sensors used to monitor a motion to be monitored of a subject while the plurality of sensors are arranged in a known direction by an arrangement mechanism such as a charging case, whereby it is possible to define the directions in which the plurality of sensors face by a common coordinate system, and thus can perform a relative calibration between the plurality of sensors. As a result, the above-described motion state monitoring system can accurately monitor the motion to be monitored of the subject. Further, in the above-described motion state monitoring system, by performing calculation processing using a trained model, it is possible to more accurately calculate whether or not a motion state of a motion to be monitored of a subject is satisfactory.

In the arrangement mechanism, both or all of the two or more sensors may be arranged so as to face in a first direction.

The arrangement mechanism may be a case so that both or all of the two or more sensors are stored so as to face in the first direction.

The case may be a charging case configured to be able to charge the two or more sensors.

The motion state monitoring system may further include a registration unit configured to at least make the two or more sensors correspond to two or more of the plurality of body parts of the subject and register them.

The motion state monitoring system may further include a determination unit configured to determine whether or not a calibration of the two or more sensors attached to the subject has been performed, in which the monitoring result generation unit may not generate a result of the monitoring of the motion to be monitored of the subject by the two or more sensors on which the calibration has not been performed.

The motion state monitoring system may further include a notification unit configured to send a notification of an error when the determination unit determines that the calibration of the two or more sensors attached to the subject has not been performed.

Each of the two or more sensors may include a magnetic attraction part as the arrangement mechanism, and the two or more sensors may be attracted to each other by the respective magnetic attraction parts thereof, so that the two or more sensors may be arranged so as to face in the first direction.

The arrangement mechanism may include: identifiers respectively assigned to the two or more sensors and distinguishable from each other by appearances thereof; and a specification unit configured to analyze the identifiers respectively assigned to the two or more sensors included in an image obtained by capturing the two or more sensors, to thereby specify a direction in which the two or more sensors face as the known direction.

A method for controlling a motion state monitoring system according to the present disclosure is a method for controlling a motion state monitoring system configured to monitor a specified motion to be monitored of a subject by two or more sensors selected from among a plurality of sensors made to correspond to a plurality of respective body parts of the subject, the method including: performing a calibration of the two or more sensors arranged in a known direction by an arrangement mechanism; and generating a result of the monitoring of the motion to be monitored of the subject based on results of detection by the two or more sensors attached to the subject after the calibration is completed. The above-described method for controlling a motion state monitoring system performs a calibration of a plurality of sensors used to monitor a motion to be monitored of a subject while the plurality of sensors are arranged in a known direction by an arrangement mechanism such as a charging case, whereby it is possible to define the directions in which the plurality of sensors face by a common coordinate system, and thus can perform a relative calibration between the plurality of sensors. As a result, the above-described method for controlling a motion state monitoring system can accurately monitor the motion to be monitored of the subject.

A control program according to the present disclosure is a control program for causing a computer to execute control processing in a motion state monitoring system configured to monitor a specified motion to be monitored of a subject by two or more sensors selected from among a plurality of sensors made to correspond to a plurality of respective body parts of the subject, the control processing including: performing a calibration of the two or more sensors arranged in a known direction by an arrangement mechanism; and generating a result of the monitoring of the motion to be monitored of the subject based on results of detection by the two or more sensors attached to the subject after the calibration is completed. The above-described control program performs a calibration of a plurality of sensors used to monitor a motion to be monitored of a subject while the plurality of sensors are arranged in a known direction by an arrangement mechanism such as a charging case, whereby it is possible to define the directions in which the plurality of sensors face by a common coordinate system, and thus can perform a relative calibration between the plurality of sensors. As a result, the above-described control program can accurately monitor the motion to be monitored of the subject.

According to the present disclosure, it is possible to provide a motion state monitoring system, a method for controlling the same, and a control program that are capable of accurately monitoring a motion state of a subject.

The above and other objects, features and advantages of the present disclosure will become more fully understood from the detailed description given hereinbelow and the accompanying drawings.

### BRIEF DESCRIPTION OF DRAWINGS

FIG. 1 is a block diagram showing an example of a configuration of a motion state monitoring system according to a first embodiment;

FIG. 2 is a diagram showing an example of body parts of a subject to which measuring instruments are to be attached;

FIG. 3 is a diagram showing an example of a configuration of the measuring instrument provided in the motion state monitoring system according to the first embodiment;

FIG. 4 is a diagram showing an example of how to attach the measuring instruments shown in FIG. 3 to a subject;

FIG. 5 is a schematic diagram showing an appearance of a charging case provided in the motion state monitoring system according to the first embodiment;

FIG. 6 is a schematic diagram showing another example of an arrangement mechanism in which a plurality of sensors on which a calibration is to be performed are arranged in a known direction;

FIG. 7 is a schematic diagram showing another example of an arrangement mechanism in which a plurality of sensors on which a calibration is to be performed are arranged in a known direction;

FIG. 8 is a flowchart showing operations performed by a motion state monitoring apparatus provided in the motion state monitoring system according to the first embodiment;

FIG. 9 is a diagram showing an example of a content displayed on a monitor;

FIG. 10 is a diagram showing an example of a content displayed on a monitor; and

FIG. 11 is a diagram showing an example of a content displayed on a monitor.

### DESCRIPTION OF EMBODIMENTS

The present disclosure will be described hereinafter through embodiments of the present disclosure. However, the disclosure according to the claims is not limited to the following embodiments. Further, all the components/structures described in the embodiments are not necessarily essential as means for solving the problem. For the clarification of the description, the following descriptions and the drawings are partially omitted and simplified as appropriate. The same elements are denoted by the same reference numerals or symbols throughout the drawings, and redundant descriptions are omitted as necessary.

### First Embodiment

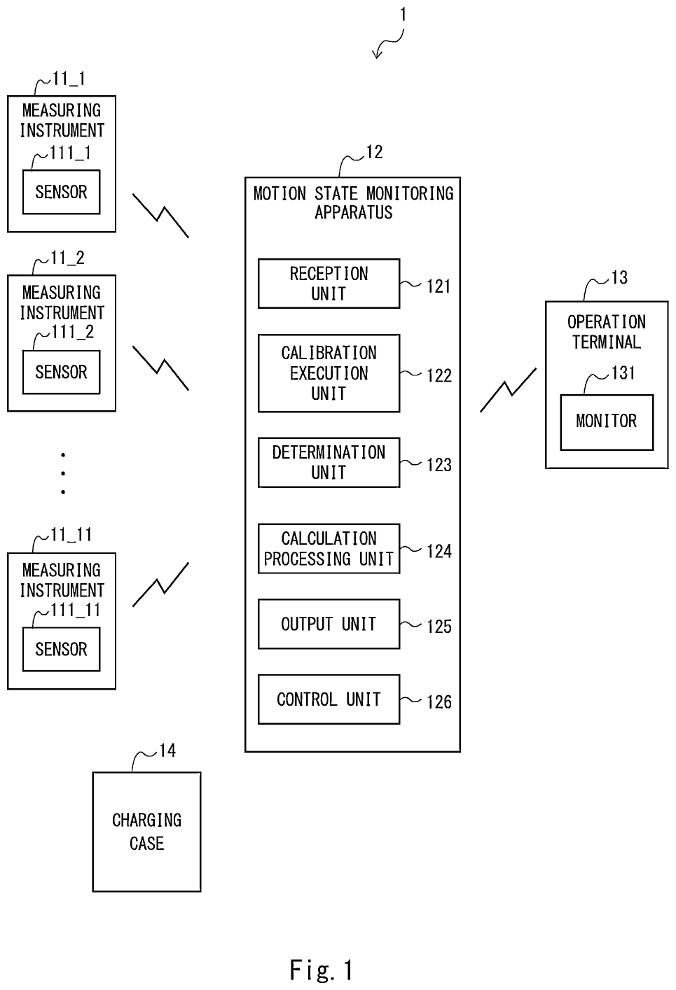

FIG. 1 is a block diagram showing a configuration example of a motion state monitoring system 1 according to a first embodiment. The motion state monitoring system 1 is a system that monitors a motion state of a subject. By a result of the monitoring, for example, a user, such as an assistant, can provide support for making the motion of the subject close to a desired motion. The details thereof will be described below.

As shown in FIG. 1, the motion state monitoring system 1 includes a plurality of measuring instruments 11, a motion state monitoring apparatus 12, and an operation terminal 13, and a charging case 14. The motion state monitoring apparatus 12 itself can be referred to as a motion state monitoring system. The motion state monitoring apparatus 12, the plurality of measuring instruments 11, and the operation terminal 13 are configured so that they can communicate with each other through a wired or wireless network. Note that, in this embodiment, an example of a case in which 11 of the measuring instruments 11 are provided will be described. In the following description, the 11 measuring instruments 11 are also referred to as measuring instruments 11_1 to 11_11, respectively, in order to distinguish them from each other.

The operation terminal 13 is a terminal capable of performing communication owned by a user or temporarily assigned to a user, such as a Personal Computer (PC) terminal, a mobile terminal such as a smartphone or a tablet terminal, or a dedicated communication terminal prepared for the motion state monitoring system 1. Note that, in this embodiment, a description will be given of a case in which the operation terminal 13 and the motion state monitoring apparatus 12 are separately provided. However, the present disclosure is not limited thereto; for example, the operation terminal 13 and the motion state monitoring apparatus 12 may be integrally formed.

For example, a user operates a monitor 131 of the operation terminal 13 by touching it with a stylus pen or a finger, or operates a mouse, a keyboard, or the like of the operation terminal 13, thereby inputting information about a subject, a result of monitoring which the user wants to display on the monitor 131, and the like to the operation terminal 13. The operation terminal 13 receives the above information and transmits it to the motion state monitoring apparatus 12 through the network. When the monitor 131 receives an operation performed by a user, the monitor 131 displays a screen for inputting information about a subject or a screen for selecting a result of monitoring which the user wants to display on the monitor 131, while when the motion state monitoring apparatus 12 has finished monitoring the motion state of the subject, the monitor 131 displays a result of monitoring received from the motion state monitoring apparatus 12.

The measuring instruments 11_1 to 11_11 are respectively attached to body parts 20_1 to 20_11 of a subject P from which motions are to be detected among various body parts of the body of the subject P, and detect the motions of the respective body parts 20_1 to 20_11 by using motion sensors (hereinafter simply referred to as sensors) 111_1 to 111_11 composed of gyro sensors, acceleration sensors, and the like. Note that the measuring instruments 11_1 to 11_11 are made to correspond to the respective body parts 20_1 to 20_11 by pairing processing performed with the motion state monitoring apparatus 12.

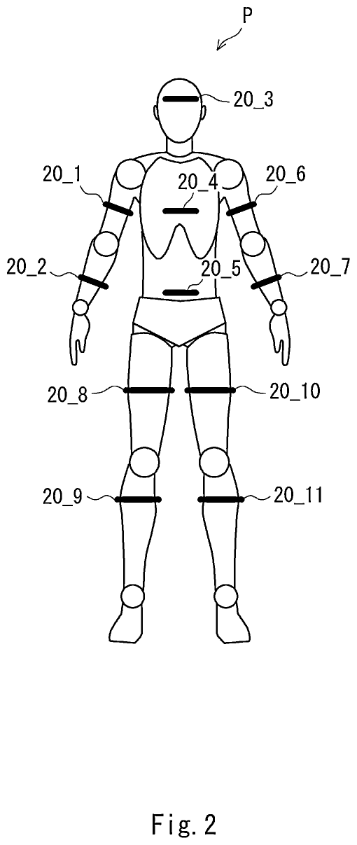

FIG. 2 is a diagram showing an example of body parts of the subject P to which the measuring instruments 11_1 to 11_11 are to be attached. In the example shown in FIG. 2, the body parts 20_1 to 20_11 to which the respective measuring instruments 11_1 to 11_11 are to be attached are a right upper arm, a right forearm, a head, a chest (a trunk), a waist (a pelvis), a left upper arm, a left forearm, a right thigh, a right lower leg, a left thigh, and a left lower leg, respectively. Note that it is not necessary to attach all of the measuring instruments 11_1 to 11_11 to the body of the subject P. It is only necessary to attach, among the measuring instruments 11_1 to 11_11, only the measuring instrument(s) required to measure a motion to be monitored (including the movement of the body part) which a user wants to monitor to the body of the subject P.

### Examples of Configurations of Measuring Instruments 11_1 to 11_11

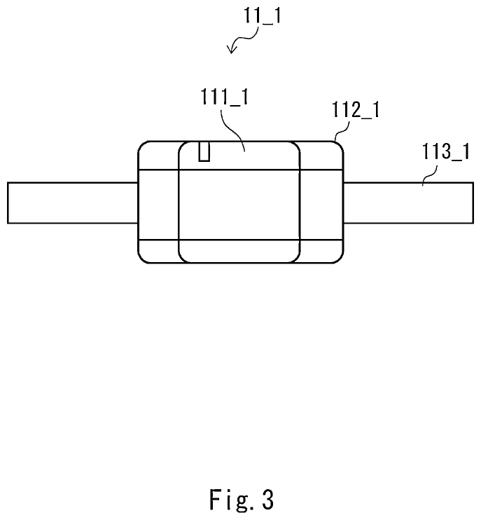

FIG. 3 is a diagram showing an example of a configuration of the measuring instrument 11_1. Note that the configuration of each of the measuring instruments 11_2 to 11_11 is similar to that of the measuring instrument 11_1, and thus the descriptions thereof will be omitted.

As shown in FIG. 3, the measuring instrument 11_1 includes the sensor 111_1, an attachment pad 112_1, and a belt 113_1. The belt 113_1 is configured so that it can be wound around the body part of the subject P from which a motion is to be detected. The sensor 111_1 is integrated with, for example, the attachment pad 112_1. The attachment pad 112_1 with which the sensor 111_1 is integrated is configured so that it can be attached to or detached from the belt 113_1.

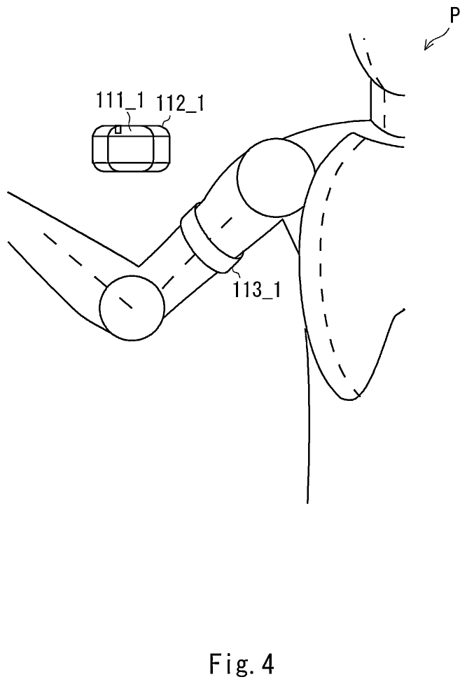

FIG. 4 is a diagram showing an example of how to attach the measuring instrument 11_1 to the subject P. In the example shown in FIG. 4, the belt 113_1 is wound around the right upper arm which is one of the body parts of the subject P from which motions are to be detected. The sensor 111_1 is attached to the belt 113_1 with the attachment pad 112_1 interposed therebetween after pairing, a calibration, and the like have been completed.

In FIG. 1, the motion state monitoring apparatus 12 outputs a result of a calculation indicating a motion state of the subject P based on results (sensing values) of detection by the sensors 111_1 to 111_11.

Specifically, the motion state monitoring apparatus 12 includes a reception unit 121, a calibration execution unit 122, a determination unit 123, a calculation processing unit 124, an output unit 125, and a control unit 126. The motion state monitoring apparatus 12 may further include a registration unit that makes the sensors 111_1 to 111_11 correspond to a plurality of the body parts of the subject P by pairing processing and registers them.

The reception unit 121 receives results of detection by the sensor 111_1 to 111_11, receive information input to the operation terminal 13 by a user, and receives information about the positions and the directions of the sensors 111_1 to 111_11 during the calibration.

The calibration execution unit 122 executes a calibration of the sensors 111_1 to 111_11. However, the calibration execution unit 122 may be configured not only to execute a calibration of all of the sensors 111_1 to 111_11, but also to execute a calibration of only the sensors attached to the body of the subject P among the sensors 111_1 to 111_11.

A calibration is, for example, processing for measuring an output value (an error component) of a sensor in a standstill state, the sensor being used to measure a motion to be monitored, and subtracting the error component from a measured value. It is assumed here that the output value of the sensor is stabilized after a predetermined period of time (about 20 seconds) has elapsed from when the sensor is brought to a standstill. In this case, in the calibration, it is desirable that the output value of the sensor after a predetermined period of time has elapsed from when the sensor is brought to a standstill be used as an error component. Therefore, in this example, the output value of the sensor after a predetermined period of time has elapsed from when a user has given an instruction to start the calibration after the sensor has been brought to a standstill is used as an error component. Further, “during the calibration” means a processing period of time until an error component is determined, and “completion of the calibration” means that the output value (the error component) of the sensor in a standstill state has been determined.

In a relative calibration between a plurality of sensors (i.e., processing of subtracting an error component of the relative position and the relative angle between a plurality of sensors from a measured value), it is necessary to define the directions in which the plurality of sensors face by a common coordinate system. Therefore, in the relative calibration between the plurality of sensors, the directions in which the plurality of sensors face needs to be known.

Therefore, the calibration execution unit 122 executes a calibration of the sensors 111_1 to 111_11 while the sensors 111_1 to 111_11 are arranged in a known direction by a predetermined arrangement mechanism.

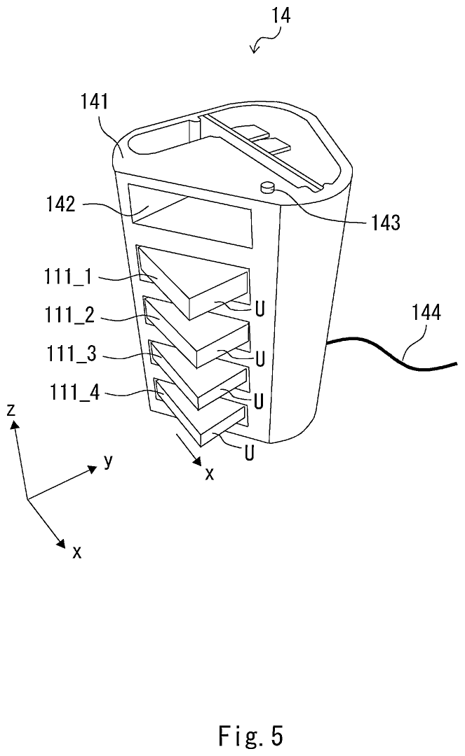

The arrangement mechanism in which a plurality of sensors on which a calibration is to be performed are arranged in a known direction is provided, for example, in the charging case 14. FIG. 5 is a schematic diagram showing an appearance of the charging case 14. Note that, in the example shown in FIG. 5, the sensors 111_1 to 111_4 are shown as a plurality of sensors on which a calibration is to be performed.

As shown in FIG. 5, the charging case 14 includes a housing 141, a plurality of storage pockets 142 formed on side surfaces of the housing 141, a switch 143, and a charging cable 144. The plurality of storage pockets 142 are arranged side by side along a vertical direction (z-axis direction), and are formed so that the sensors 111_1 to 111_11 can be stored while their upper surfaces face in a predetermined direction (x-axis direction). In the example shown in FIG. 5, the sensors 111_1 to 111_4 are stored in the four respective storage pockets 142 of the charging case 14. Thus, the directions in which the sensors 111_1 to 111_4 face have become the same, that is, become known. When the switch 143 is pressed by a user, the calibration execution unit 122 determines that the sensors 111_1 to 111_4 are arranged in a known direction and then starts a calibration of the sensors 111_1 to 111_4. Further, at this time, the sensors 111_1 to 111_4 stored in the respective four storage pockets 142 of the charging case 14 are charged through the charging cable 144.

In this embodiment, an example of case where an arrangement mechanism in which a plurality of sensors on which a calibration is to be performed are arranged in a known direction is provided in the charging case 14 has been described. However, the present disclosure is not limited to this case. The arrangement mechanism may be provided in a case having no charging function instead of being provided in the charging case 14.

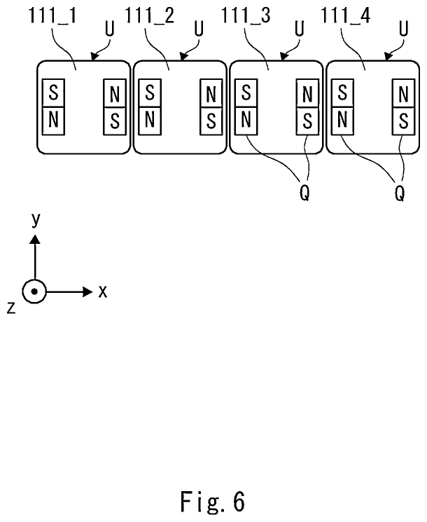

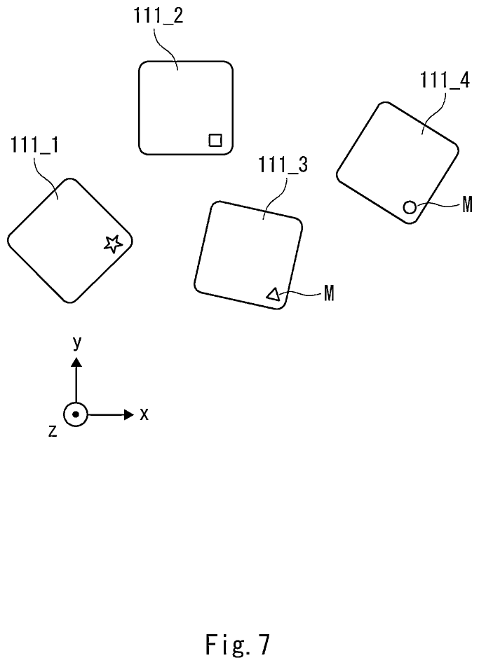

Further, an arrangement mechanism in which a plurality of sensors on which a calibration is to be performed are arranged in a known direction may be provided in a place other than a case such as a charging case. Examples thereof will be described below with reference to FIGS. 6 and 7.

FIG. 6 is a schematic diagram showing another example of an arrangement mechanism in which a plurality of sensors on which a calibration is to be performed are arranged in a known direction. In the example shown in FIG. 6, a pair of magnetic attraction parts Q are attached to both ends of each of the sensors 111_1 to 111_4. In other words, each of the sensors 111_1 to 111_4 includes the pair of magnetic attraction parts Q as the arrangement mechanism. The sensors 111_1 to 111_4 are attracted to each other by the respective magnetic attraction parts Q thereof, whereby the sensors 111_1 to 111_4 are arranged so that their upper surfaces face in the same direction. That is, the directions of the sensors 111_1 to 111_4 have become known.

FIG. 7 is a schematic diagram showing another example of an arrangement mechanism in which a plurality of sensors on which a calibration is to be performed are arranged in a known direction. In the example shown in FIG. 7, each of the sensors 111_1 to 111_4 is provided with a marker M as an example of an identifier distinguishable from the appearance thereof. In this case, the motion state monitoring apparatus 12 includes a specification unit that captures, for example, the sensors 111_1 to 111_4 randomly arranged on a desk by a camera and analyzes the marker M provided to each of the sensors 111_1 to 111_4 included in the captured image, thereby specifying a direction in which each of the sensors 111_1 to 111_4 faces as a known direction. In other words, in the example shown in FIG. 7, an arrangement mechanism (a mechanism of arrangement) in which a plurality of sensors on which a calibration is to be performed are arranged in a known direction is implemented by an identifier such as a marker assigned to each of the sensors and the above-described specification unit. Note that the identifier distinguishable from the appearance thereof is not limited to the marker M, and may instead be a pattern, a QR code (registered trademark), or the like.

The determination unit 123 determines whether or not a calibration of the sensor attached to the subject P has been executed before the sensor is attached to the subject P. For example, the determination unit 123 determines that a calibration has been executed by receiving a notification that the calibration has been executed from the calibration execution unit 122. Then the determination unit 123 determines whether or not a calibration of the sensor attached to the subject P has been executed before the sensor is attached to the subject P by comparing the time when the calibration of the sensor is completed with the time when the sensor is attached to the subject P.

The calculation processing unit 124 performs calculation processing based on a result of detection by each of the sensors 111_1 to 111_11 on which a calibration has been executed and generates a result of the calculation indicating a motion state of a motion to be monitored of the subject P. Therefore, the calculation processing unit 124 may be referred to as a monitoring result generation unit that generates a result of the monitoring of the motion to be monitored of the subject P. Examples of the motion to be monitored include motions such as bending and stretching of the right shoulder, adduction and abduction of the right shoulder, internal and external rotation of the right shoulder, bending and stretching of the right elbow, pronation and supination of the right forearm, bending and stretching of the head, rotation of the head, bending and stretching of the chest and the waist, rotation of the chest and the waist, lateral bending of the chest and the waist, bending and stretching of the left shoulder, adduction and abduction of the left shoulder, internal and external rotation of the left shoulder, bending and stretching of the left elbow, and pronation and supination of the left forearm. Further, the motion to be monitored includes movement of the body part to which a sensor is attached. The motion to be monitored also includes, for example, an angle of the joint of the body of the subject P to be measured based on results of the detection by a plurality of sensors and an angle of the joint in any coordinate system to be measured based on a result of the detection by one of the sensors. In the following description, generation of a result of calculation indicating a motion state of a motion to be monitored is also referred to as measurement of a motion to be monitored.

For example, the calculation processing unit 124 performs calculation processing based on a result of detection by each of the sensor 111_1 attached to the right upper arm (the body part 20_1) and the sensor 111_2 attached to the right forearm (the body part 20_2) of the subject P among the sensors 111_1 to 111_11, and generates a result of the calculation indicating a motion state of the bending and stretching motion of the right elbow of the subject P.

Alternatively, the calculation processing unit 124 performs calculation processing based on a result of detection by each of the sensor 111_5 attached to the waist (the body part 20_5) and the sensor 111_8 attached to the right thigh (the body part 20_8) of the subject P among the sensors 111_1 to 111_11, and generates a result of the calculation indicating a motion state of the lateral bending motion of the right side of the waist of the subject P.

Note that when the determination unit 123 determines that the calibration of the sensor attached to the subject P has not been executed before the sensor is attached to the subject P, the calculation processing unit 124 may be configured so as not to generate a result of the calculation indicating a motion state of the motion to be monitored of the subject P. Alternatively, when the determination unit 123 determines that the calibration of the sensor attached to the subject P has not been executed before the sensor is attached to the subject P, the calculation processing unit 124 may be configured so as to send a notification of an error.

Note that the calculation processing unit 124 may perform calculation processing by using a trained model generated by machine learning using a result of the past detection of the sensor. By performing calculation processing using the trained model, the calculation processing unit 124 can more accurately calculate whether or not a motion state of a motion to be monitored of the subject P is satisfactory.

The output unit 125 outputs a result of the calculation by the calculation processing unit 124. The information (the result of the calculation, error information) output from the output unit 125 is transferred to the operation terminal 13 through the network, visualized in the form of a graph or the like, and displayed on the monitor 131 of the operation terminal 13. As a result, a user can know the motion state of the motion to be monitored of the subject P, and can, for example, use it for assisting the subject P.

In order to make a user select a result of the measurement of the motion to be monitored to be displayed on the monitor 131, the control unit 126 displays an icon display area S1 and display setting areas A1 to A3 on a screen of the monitor 131. Note that the number of display setting areas is not limited to three, and may be one or more. Further, the control unit 126 displays a measurement result icon indicating a result of measurement of the motion to be monitored that can be displayed on the monitor 131 in the icon display area S1. In other words, the control unit 126 displays a measurement result icon indicating a result of measurement of the motion to be monitored that can be acquired from the sensor attached to the subject P. A user can select, from among the measurement result icons displayed in the icon display area S1, a measurement result icon corresponding to a result of the measurement of the motion to be monitored which the user wants to display on the monitor 131.

The reception unit 121 accepts a setting operation performed by a user for one of the display setting areas A1 to A3 for setting areas that display the details of the results of measurement for the measurement result icons displayed in the icon display area S1. For example, a user moves the measurement result icon displayed in the icon display area S1 to the display setting area A1. Specifically, a user drags and drops the measurement result icon displayed in the icon display area S1 onto the display setting area A1 by a mouse operation, a touch operation, or the like. By doing so, the reception unit 121 accepts a setting operation performed by a user for the display setting area A1 for setting an area that displays the details of the result of measurement for the measurement result icon. As a result, the display setting area A1 displays the details of the selected result of measurement (e.g., a graphed result of measurement).

Note that, in this embodiment, although a description has been given of an example of a case in which the reception unit 121 accepts a setting operation performed by a user for the display setting area A1 for setting an area that displays the details of a result of measurement for one measurement result icon displayed in the icon display area S1, the present disclosure is not limited to this case. The reception unit 121 may further accept a setting operation performed by a user for one of the display setting areas A2 and A3 for setting areas that display the details of a result of measurement for another measurement result icon displayed in the icon display area S1. In this case, for example, a user moves one measurement result icon displayed in the icon display area S1 to the display setting area A1 and moves another measurement result icon displayed in the icon display area S1 to one of the display setting areas A2 and A3. By doing so, the reception unit 121 accepts a setting operation performed by a user for the display setting area A1 for setting an area that displays the details of a result of measurement for one measurement result icon and a setting operation performed by the user to one of the display setting areas A2 and A3 for setting areas that display the details of a result of measurement for another measurement result icon. As a result, the details of one selected result of measurement are displayed in the display setting area A1 and the details of another selected result of measurement are displayed in one of the display setting areas A2 and A3.

Further, in this embodiment, although a description has been given of an example of a case in which the reception unit 121 accepts a setting operation performed by a user for the display setting area A1 for setting an area that displays the details of a result of measurement for one measurement result icon displayed in the icon display area S1, the present disclosure is not limited to this case. The reception unit 121 may accept a setting operation performed by a user for the display setting area A1 for setting an area that displays the details of a result of measurement for another measurement result icon displayed in the icon display area S1. In this case, for example, a user moves two measurement result icons displayed in the icon display area S1 to the display setting area A1. By doing so, the reception unit 121 accepts a setting operation performed by a user for the common display setting area A1 for setting an area that displays the details of a result of measurement for the two measurement result icons. As a result, the display setting area A1 displays the details of the two selected results of measurement. For example, two graphed results of measurement are displayed in the display setting area A1 while they are superimposed on each other.

Further, the reception unit 121 may accept a change operation for changing an area that displays the result of measurement corresponding to the measurement result icon from the display setting area A1 to one of the display setting areas A2 and A3 in response to, for example, a user moving (dragging and dropping) the measurement result icon from the display setting area A1 onto one of the display setting areas A2 and A3. Further, the measurement result icon set in the display setting area A1 may be set in one or both of the display setting areas A2 and A3 at the same time.

### Operation of Motion State Monitoring Apparatus 12

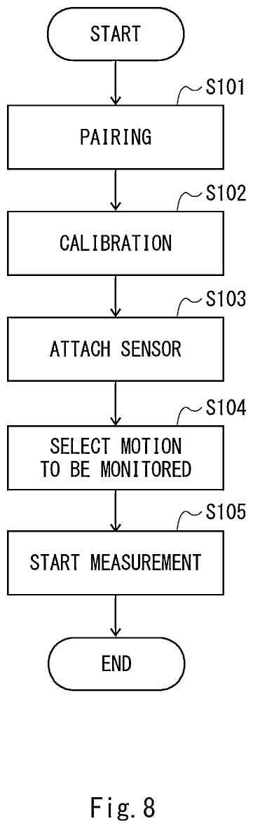

Next, operations performed by the motion state monitoring apparatus 12 will be described with reference to FIG. 8. FIG. 8 is a flowchart showing the operations performed by the motion state monitoring apparatus 12.

First, the motion state monitoring apparatus 12 performs pairing processing between the motion state monitoring apparatus 12 and each of the measuring instruments 11_1 to 11_11, thereby making the measuring instruments 11_1 to 11_11 correspond to the body parts 20_1 to 20_11 of the subject P (Step S101).

After that, the motion state monitoring apparatus 12 performs a calibration of the sensors 111_1 to 111_11 (Step S102). Note that, during the calibration, for example, the sensors 111_1 to 111_11 are stored in the charging case 14, so that they are arranged in a known direction. Thus, the motion state monitoring apparatus 12 can define the directions in which the sensors 111_1 to 111_11 face by a common coordinate system, and thus can perform a relative calibration between the sensors 111_1 to 111_11.

During the calibration, the monitor 131 displays, for example, the information that “Calibration is in progress. Place the sensor on the desk and do not move it”. Upon completion of the calibration, the monitor 131 displays, for example, the information that “Calibration has been completed. Attach the sensor”. Note that the information indicating that the calibration is in progress or that the calibration has been completed is not limited to being given by displaying it on the monitor 131, and may instead be given by other notification methods such as by voice. Further, the order in which the calibration processing and the pairing processing are performed may be reversed.

After the calibration has been completed, the sensor is attached to the subject P (Step S103). In this example, among the sensors 111_1 to 111_11, the sensors 111_1, 111_2, 111_5, and 111_8 are attached to the right upper arm (the body part 20_1), the right forearm (the body part 20_2), the waist (the body part 20_5), and the right thigh (the body part 20_8) of the subject P, respectively.

After that, the motion state monitoring apparatus 12 measures a motion to be monitored that can be measured using the sensors attached to the subject P among a plurality of motions to be monitored (Step S104).

After that, the motion state monitoring apparatus 12 outputs a result of the measurement (a result of the calculation indicating a motion state of the motion to be monitored of the subject P) (Step S105).

Specifically, the motion state monitoring apparatus 12 first displays the icon display area S1 and the display setting areas A1 to A3 on the screen of the monitor 131. Note that the number of display setting areas is not limited to three, and may be one or more. Further, the motion state monitoring apparatus 12 displays a measurement result icon indicating a result of measurement of the motion to be monitored that can be displayed on the monitor 131 in the icon display area S1. In other words, the motion state monitoring apparatus 12 displays a measurement result icon indicating a result of measurement of the motion to be monitored that can be acquired from the sensor attached to the subject P in the icon display area S1. A user can select, from among the measurement result icons displayed in the icon display area S1, a measurement result icon corresponding to a result of measurement of the motion to be monitored which the user wants to display on the monitor 131.

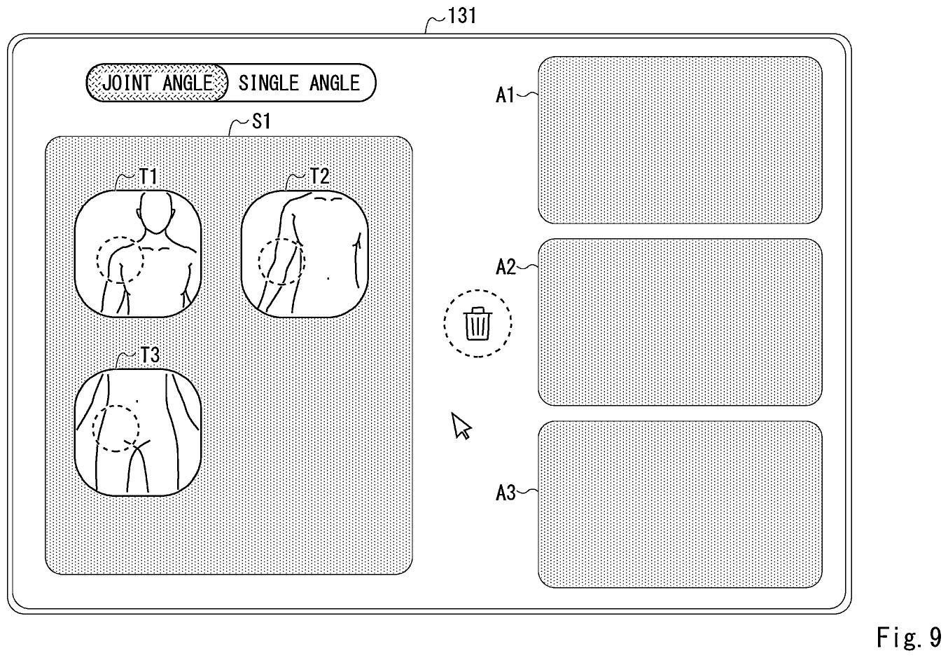

In the example shown in FIG. 9, the icon display area S1 is displayed on the left side of the screen of the monitor 131, while the display setting areas A1 to A3 are displayed on the right side of the screen of the monitor 131. Further, the icon display area S1 displays a measurement result icon T1 indicating a result of measurement of the “adduction and abduction motion of the right shoulder” that can be acquired from the sensor 111_1, a measurement result icon T2 indicating a result of measurement of the “bending and stretching motion of the right elbow” that can be acquired from the sensors 111_1 and 111_2, and a measurement result icon T3 indicating a result of measurement of the “lateral bending motion of the right side of the waist” that can be acquired from the sensors 111_5 and 111_8. That is, in the example shown in FIG. 9, a user can select, from among the three measurement result icons T1 to T3 displayed in the icon display area S1, a measurement result icon corresponding to a result of the measurement of the motion to be monitored which the user wants to display on the monitor 131.

After that, the motion state monitoring apparatus 12 accepts a setting operation performed by a user for one of the display setting areas A1 to A3 for setting areas that display the details of the results of measurement for the measurement result icons T1 to T3 displayed in the icon display area S1.

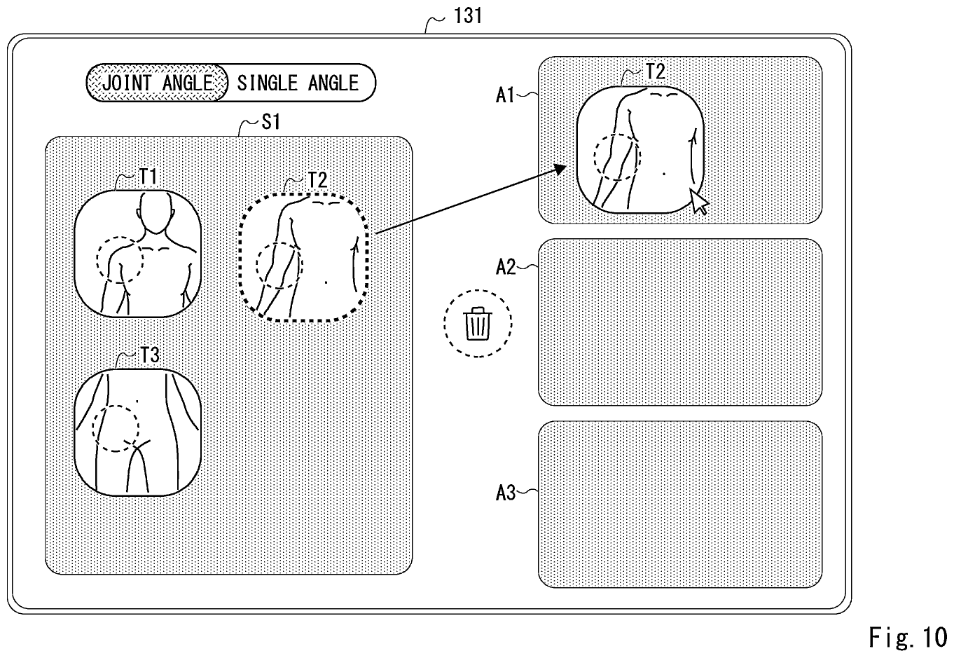

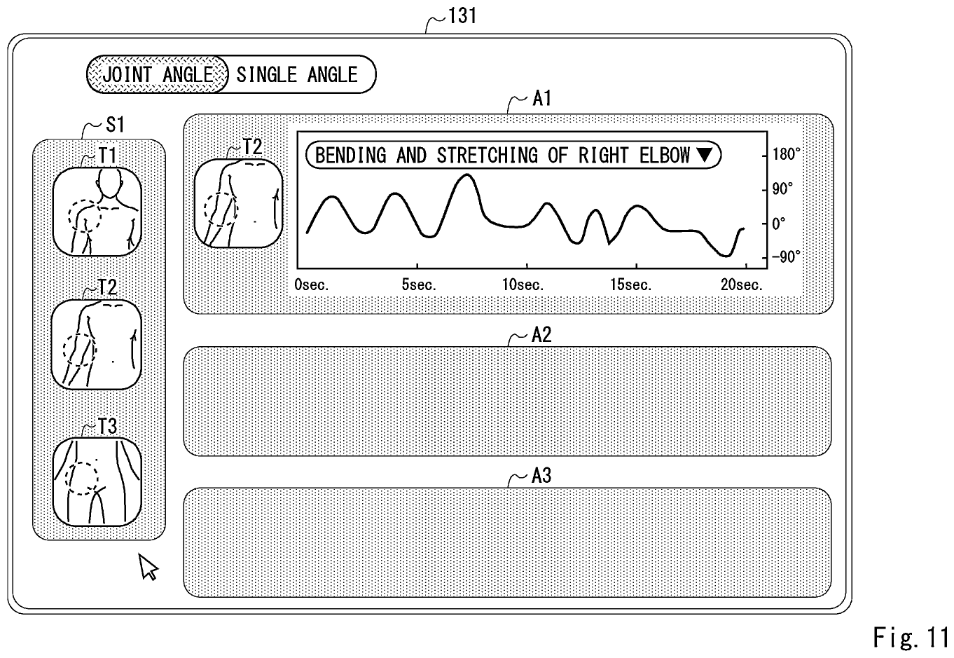

In the example shown in FIG. 10, a user drags and drops the measurement result icon T1 among the measurement result icons T1 to T3 displayed in the icon display area S1 onto the display setting area A1 by a mouse operation, a touch operation, or the like. By doing so, the motion state monitoring apparatus 12 accepts a setting operation performed by a user for the display setting area A1 for setting an area that displays the details of the result of measurement for the measurement result icon T1. As a result, as shown in the example of FIG. 11, the display setting area A1 displays the details of the result of measurement (e.g., a graphed result of measurement) corresponding to the measurement result icon T1. Note that, even when the measurement result icon T1 is displayed in the display setting area A1 by a drag-and-drop operation, it is still also displayed in the icon display area S1. However, the measurement result icon T1 may be deleted from the icon display area S1 in response to the display of the measurement result icon T1 in the display setting area A1 by a drag-and-drop operation.

As described above, the motion state monitoring system 1 according to this embodiment performs a calibration of a plurality of sensors used to monitor a motion to be monitored while the plurality of sensors are arranged in a known direction by being stored in the charging case 14 or the like. Thus, the motion state monitoring system 1 according to this embodiment can define the directions in which the plurality of sensors face by a common coordinate system, and thus can perform a relative calibration between the plurality of sensors. As a result, the motion state monitoring system 1 according to this embodiment can accurately monitor the motion to be monitored of the subject.

Further, in the present disclosure, it is possible to implement some or all of the processes performed in the motion state monitoring system 1 by causing a Central Processing Unit (CPU) to execute a computer program.

The above-described program includes instructions (or software codes) that, when loaded into a computer, cause the computer to perform one or more of the functions described in the example embodiments. The program may be stored in a non-transitory computer readable medium or a tangible storage medium. By way of example, and not a limitation, non-transitory computer readable media or tangible storage media can include a Random-Access Memory (RAM), a Read-Only Memory (ROM), a flash memory, a Solid-State Drive (SSD) or other types of memory technologies, a CD-ROM, a Digital Versatile Disc (DVD), a Blu-ray (Registered Trademark) disc or other types of optical disc storage, a magnetic cassette, a magnetic tape, and a magnetic disk storage or other types of magnetic storage devices. The program may be transmitted on a transitory computer readable medium or a communication medium. By way of example, and not a limitation, transitory computer readable media or communication media can include electrical, optical, acoustical, or other forms of propagated signals.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A motion state monitoring system configured to monitor a specified motion to be monitored of a subject by two or more sensors selected from among a plurality of sensors made to correspond to a plurality of respective body parts of the subject, the motion state monitoring system comprising:
an arrangement mechanism in which the two or more sensors are arranged in a known direction;
a calibration execution unit configured to perform a calibration of the two or more sensors arranged in the known direction by the arrangement mechanism; and
a monitoring result generation unit configured to generate a result of the monitoring of the motion to be monitored of the subject based on results of detection by the two or more sensors attached to the subject after the calibration is completed.

2. The motion state monitoring system according to claim 1, wherein in the arrangement mechanism, both or all of the two or more sensors are arranged so as to face in a first direction.

3. The motion state monitoring system according to claim 2, wherein the arrangement mechanism is provided in a case so that both or all of the two or more sensors are stored so as to face in the first direction.

4. The motion state monitoring system according to claim 3, wherein the case is a charging case configured to be able to charge the two or more sensors.

5. The motion state monitoring system according to claim 1, further comprising a registration unit configured to at least make the two or more sensors correspond to two or more of the plurality of body parts of the subject and register them.

6. The motion state monitoring system according to claim 1, further comprising a determination unit configured to determine whether or not a calibration of the two or more sensors attached to the subject has been performed,
wherein the monitoring result generation unit does not generate a result of the monitoring of the motion to be monitored of the subject by the two or more sensors on which the calibration has not been performed.

7. The motion state monitoring system according to claim 6, further comprising a notification unit configured to send a notification of an error when the determination unit determines that the calibration of the two or more sensors attached to the subject has not been performed.

8. The motion state monitoring system according to claim 2, wherein
each of the two or more sensors comprises a magnetic attraction part as the arrangement mechanism, and
the two or more sensors are attracted to each other by the respective magnetic attraction parts thereof, so that the two or more sensors are arranged so as to face in the first direction.

9. The motion state monitoring system according to claim 1, wherein the arrangement mechanism comprises:
identifiers respectively assigned to the two or more sensors and distinguishable from each other by appearances thereof; and
a specification unit configured to analyze the identifiers respectively assigned to the two or more sensors included in an image obtained by capturing the two or more sensors, to thereby specify a direction in which the two or more sensors face as the known direction.

10. A method for controlling a motion state monitoring system configured to monitor a specified motion to be monitored of a subject by two or more sensors selected from among a plurality of sensors made to correspond to a plurality of respective body parts of the subject, the method comprising:
performing a calibration of the two or more sensors arranged in a known direction by an arrangement mechanism; and
generating a result of the monitoring of the motion to be monitored of the subject based on results of detection by the two or more sensors attached to the subject after the calibration is completed.

11. A non-transitory computer readable medium storing a control program for causing a computer to execute control processing in a motion state monitoring system configured to monitor a specified motion to be monitored of a subject by two or more sensors selected from among a plurality of sensors made to correspond to a plurality of respective body parts of the subject, the control processing comprising:
performing a calibration of the two or more sensors arranged in a known direction by an arrangement mechanism; and
generating a result of the monitoring of the motion to be monitored of the subject based on results of detection by the two or more sensors attached to the subject after the calibration is completed.
